(19) United States
(12) Patent Application Publication
(10) Pub. No.: US 2022/0253160 A1
(43) Pub. Date: Aug. 11, 2022

(54) THERMOELECTRIC FEEDBACK MOUSE

(71) Applicant: Hewlett-Packard Development Company, L.P., Spring, TX (US)

(72) Inventors: Alexander Williams, Spring, TX (US); Hsiao-Jou Lin, Spring, TX (US); Anthony Kaplanis, Spring, TX (US); John Frederick, Spring, TX (US)

(73) Assignee: Hewlett-Packard Development Company, L.P., Spring, TX (US)

(21) Appl. No.: 17/628,937

(22) PCT Filed: Sep. 30, 2019
(86) PCT No.: PCT/US2019/053766

§ 371 Date: Jan. 21, 2022

(51) Int. Cl. G06F 3/0354; G06F 1/20; G01K 1/143; G05D 23/19

(52) U.S. Cl. CPC G06F 3/03543; G06F 1/206; A61B 5/01; G05D 23/1931; G01K 1/143

(57) ABSTRACT

In an example implementation according to aspects of the present disclosure, a mouse system comprising a plurality of temperature sensors, a thermoelectric device, and a processor. The processor receives a first input and second input from a first temperature sensor and second temperature sensor respectively. The first temperature sensor is in proximity to a users fingers, and the second temperature sensor is in proximity to a users palm. The processor activates the thermoelectric device based on the received first and second inputs. The processor receives a feedback from a user response and the feedback, the first input and second input are provided as input to a machine learning model.

### BACKGROUND

A computer mouse may be used as a pointing device. The computer mouse may receive inputs in the form of detected clicks and motion from a user and allow a user to interact with a computing device.

### BRIEF DESCRIPTION OF THE DRAWINGS

FIG. 1 is a block diagram illustrating a thermoelectric feedback mouse system, according to an example:

FIG. 2 is a flow diagram illustrating a method implementing a thermoelectric feedback mouse system, according to an example; and

FIG. 3 is a computing device for geospatial display configuration, according to an example.

### DETAILED DESCRIPTION

People today are spending increasing amount of time on personal computers For some users, especially with poor circulation, this prolonged usage can cause discomfort by their hands getting cold. Another subset of users has an issue with their hands getting too hot, especially when playing games. A thermoelectric cooling (TEC) device integrated into the mouse may generate heat or cooling. Utilizing a TEC may a create a situation that may leave a user's hand too hot or too cold. Disclosed herein is a thermoelectric feedback mouse system that utilizes a plurality of temperature sensors with a TEC to create a temperature gradient, to which a machine learning model is trained to a specific user.

In one example, a plurality of temperature sensors, a TEC, and a processor, receive temperature values from a proximity near a user's fingers and palm, receive a user feedback, activate the TEC device, and input the temperature readings and the feedback into a machine learning model.

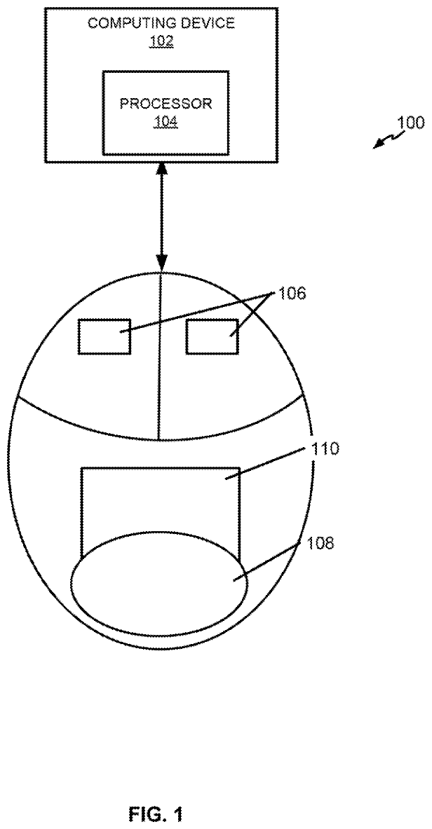

FIG. 1 is a block diagram illustrating a thermoelectric feedback mouse system 100, according to an example. The thermoelectric feedback mouse system 100 may include a computing device 102, a processor 104, a plurality of sensors, including a first temperature sensor 106 and a second temperature sensor 108, and a thermoelectric cooling device 110.

A computing device 102 may house the processor 104 in one example. The computing device 102 may include but is not limited to a personal computer, laptop computer, cloud enabled gaming system, or a video game console. In another implementation the processor 104 may be housed in the mouse or pointing device. The processor 104 may be a specific purpose processor or tensor processing unit designed to receive the temperature sensor input, activate the thermoelectric device, and implement a machine learning model on the mouse. The computing device 102 may include a communication channel that may transmit mouse inputs to the computing device 102 to be processed by the processor 104. The computing device 102 may provide power to operate the plurality of sensors and the thermoelectric cooling device 110. The communication channel may be implemented as a universal serial bus (USB) cable. In another implementation, the communication channel may be wireless utilizing a radio frequency receiver interfacing with the computing device via USB. The mouse or pointing device may include a radio transceiver and independent power supply to operate the plurality of temperature sensors and the thermoelectric cooling device 110. The mouse or pointing device may include a housing that supports the physical components including the plurality of temperature sensors, a thermoelectric cooling device, as well as the mouse internals (not shown).

A plurality of temperature sensors including a first temperature sensor 106 and a second temperature sensor 108. The plurality of temperature sensors may be implemented as but not limited to negative temperature coefficient thermistors, resistance temperature detector, thermocouple, or semiconductor-based temperature-sensitive voltage circuits. In another implementation, additional sensors may be utilized to provide additional temperature inputs. An additional sensor may include an ambient temperature sensor. The ambient temperature sensor may include a connected smart thermostat. Input from the ambient temperature sensor may be received from additional systems (no shown) that may interface with the computing device 102 and the processor 104.

The processor 104 may be the central processing unit (CPU) of the host computing device 102. In another example, the processor 104 may be virtualized and distributed across more than one general purpose processors. In another implementation, the processor 104 may be a graphics processing unit (GPU) utilized to execute parallel machine learning models. In another implementation, the processor 104 may be a dedicated application-specific integrated circuit (ASIC) dedicated to machine learning activities such as a tensor processing unit (TPU).

In another implementation, the thermoelectric feedback mouse may include a biometric sensor. The biometric sensor may include a fingerprint sensor integrated into the surface of the mouse. The fingerprint sensor may be aligned such that a user's fingertip interfaces with the fingerprint sensor. The fingerprint sensor may correlate a first input and second input from the plurality of temperatures sensors to a specific user. Correlating the sensor input may be utilized to train separate machine learning models based on specific users. For example, a family may share a computer with an attached thermoelectric feedback mouse. The mother of the family may use the mouse and may define comfort as one temperature range. The father of the family may define his comfort as a different temperature range. Utilizing the fingerprint scanner, and a scanned finger print, the thermoelectric feedback mouse may correlate respective temperature inputs, and feedbacks to that specific user.

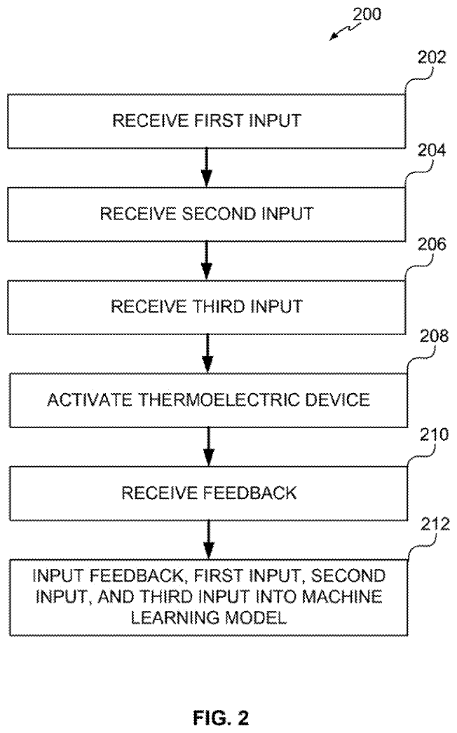

FIG. 2 is a flow diagram illustrating a method implementing a thermoelectric feedback mouse system, according to an example.

At 202, the processor 104 receives a first input from a first temperature sensor of a plurality of temperature sensors wherein the first temperature sensor is positioned in proximity to a user's finger. The first input may correspond to a temperature reading from a user's finger. In another implementation, the first temperature sensor may correspond to a logical grouping of two or more sensors located in proximity to a user's fingers on the mouse or pointing device. Each of the two or more sensors may provide a temperature reading to be included in the first input. The first temperature sensor transmits the first input to the processor 104 over a communication channel which may be wired or wireless.

At 204, the processor 104 receives a second input from a second temperature sensor from the plurality of temperature sensors wherein the first temperature sensor is positioned in proximity to a user's palm. The second input may correspond to a temperature reading from a user's palm. In another implementation, the second temperature sensor may correspond to a logical grouping of two or more sensors located in proximity to a user's palm on the mouse or pointing device. Each of the two or more sensors may provide a temperature reading to be included in the second input. The second temperature sensor transmits the second input to the processor 104 over a communication channel which may be wired or wireless.

At 206, the processor 104 receives a third input from an ambient temperature sensor from the plurality of temperature sensors. As discussed previously, the ambient temperature sensor may be implemented as a connected smart thermostat. The processor 104 may access the ambient temperature sensor through an application programming interface (API) and collect information corresponding to the ambient temperature of the physical location. Utilizing an ambient temperature sensor that is not integrated with the mouse system may provide more accurate ambient temperatures, as the thermoelectric cooling device may generate dissipate heat when cooling the user's hand. The heat dissipation may interfere with any local measurements of ambient temperature.

At 208, the processor 104 activates a thermoelectric device responsive to a machine learning model output wherein the first input, second input, and third input comprise a corresponding machine learning model input. The first, second, and third inputs may be utilized inputs into a classification model. The machine learning model may be a linear regression, multi-class classification or a support vector machine. The inputs result in an output classification indicating comfort or discomfort. During any period between temperature polling, the classification may remain indicating comfort, and the processor 104 may keep the thermoelectric cooling device activated.

At 210, the processor 104 receives feedback from user responsive to the activation of the thermoelectric device. The feedback may be a temperature adjustment Upon reaching a point of discomfort, the user may provide the temperature adjustment to the mouse system. The user feedback may be used as a classification to train the machine learning as which combinations of the first input, second input, and third input equates to user comfort and discomfort. As such, the machine learning model may activate the thermoelectric cooling device while the input corresponds to a “comfort” classification. Once the classification changes, based on inputs, to “discomfort” the machine learning model may deactivate the thermoelectric device.

At 212, the processor 104 inputs feedback, first input, second input, and third input into the machine learning model. The combination of the first input, second input, third input and the feedback may be input into the machine learning model as training data to classify the discomfort.

In another implementation, the processor 104 determine a temperature gradient between the first input and the second input. Additionally, temperature gradients based on the first input and second input may be calculated and provided as additional data points for the machine learning model. These inputs may be determinative for classification output of the machine learning model. In another example, with a mouse or point device may include more than one individually controllable thermoelectronic cooling devices within the mouse. Additionally, more corresponding sensors may be implemented across various locations on the surface of the mouse. A gradient map of the hand may be generated based on difference recorded at each of the sensor locations and the thermoelectric cooling devices may be be individually activated to generate heat or cooling to any part of the gradient map that may be above or below the comfort range.

In this implementation, the processor 104 inputs the temperature gradient, feedback, first input and second input into the machine learning model. By providing the additional data point of the temperature gradient, the machine learning model may be able to be trained to be more accurate in classifying combination of temperatures as inputs.

The processor 104 inputs the ambient temperature, the feedback, first input and second input into the machine learning model. In this implementation, ambient temperature may become a determinative factor in the classification of comfort versus discomfort. The ambient temperature may be determinative because fingers with bad circulation may feel colder to a user when the ambient temperature is lower; thereby decreasing comfort. A user may activate or deactivate the thermoelectric cooling device based on the ambient temperature in conjunction with the temperatures recorded on the mouse system itself.

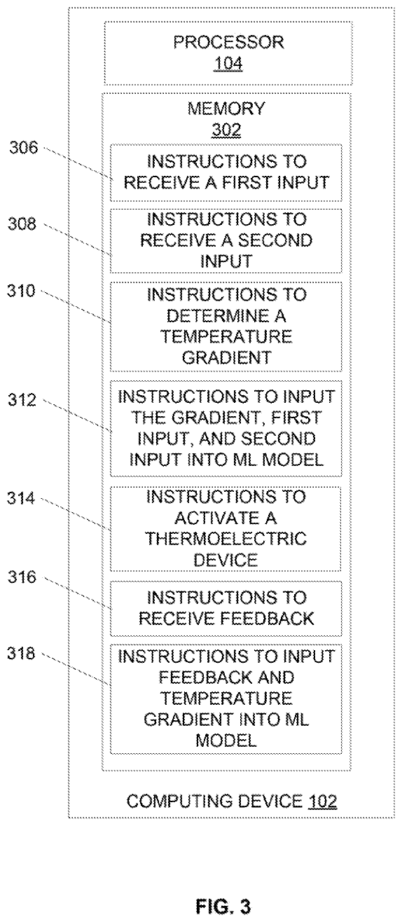

FIG. 3 is a computing device 102 for supporting a thermoelectric feedback mouse system, according to an example. The computing device 102 depicts a processor 104 and a memory 302 and, as an example of the computing device 102 for geospatial display configuration, the memory 302 may include instructions 306-318 that are executable by the processor 104. The processor 104 may be synonymous with the embedded processors found in common computing environments including central processing units (CPUs). In another implementation the processor 104 may be an embedded microcontroller for processing inputs. The memory 302 can be said to store program instructions that, when executed by processor 104, implement the components of the computing device 102. The executable instructions may correspond to computer implemented instructions corresponding to the method of FIG. 2. The executable program instructions stored in the memory 302 include, as an example, instructions to receive a first input 306, instructions to receive a second input 308, instructions to determine a temperature gradients 310, instructions to input the gradient, first input, and second input into a machine learning model 312, instructions to activate a thermoelectric device 314, instructions to receive feedback from a user 316, instructions to input the feedback and temperature gradient into the machine learning model 318.

Memory 302 represents generally any number of memory components capable of storing instructions that can be executed by processor 104. Memory 302 is non-transitory in the sense that it does not encompass a transitory signal but instead is made up of at least one memory component configured to store the relevant instructions. As a result, the memory 302 may be a non-transitory computer-readable storage medium. Memory 302 may be implemented in a single device or distributed across devices. Likewise, processor 104 represents any number of processors capable of executing instructions stored by memory 302. Processor 104 may be integrated in a single device or distributed across devices. Further, memory 302 may be fully or partially integrated in the same device as processor 104, or it may be separate but accessible to that device and processor 104.

In one example, the program instructions 306-318 can be part of an installation package that, when installed, can be executed by processor 104 to implement the components of the computing device 102. In this case, memory 302 may be a portable medium such as a CD, DVD, or flash drive, or a memory maintained by a server from which the installation package can be downloaded and installed. In another example, the program instructions may be part of an application or applications already installed. In another example, the memory 302 may be internal flash memory to an input device, wherein the program instructions 308-318 may be installed from the input device manufacturer. Here, memory 302 may include integrated memory such as a flash ROM, solid state drive, or the like.

It is appreciated that examples described may include various components and features. it is also appreciated that numerous specific details are set forth to provide a thorough understanding of the examples. However, it is appreciated that the examples may be practiced without limitations to these specific details. In other instances, well known methods and structures may not be described in detail to avoid unnecessarily obscuring the description of the examples. Also, the examples may be used in combination with each other.

Reference in the specification to “an example” or similar language means that a particular feature, structure, or characteristic described in connection with the example is included in at least one example, but not necessarily in other examples. The various instances of the phrase “in one example” or similar phrases in various places in the specification are not necessarily all referring to the same example.

It is appreciated that the previous description of the disclosed examples is provided to enable any person skilled in the art to make or use the present disclosure. Various modifications to these examples will be readily apparent to those skilled in the art, and the generic principles defined herein may be applied to other examples without departing from the scope of the disclosure. Thus, the present disclosure is not intended to be limited to the examples shown herein but is to be accorded the widest scope consistent with the principles and novel features disclosed herein.

What is claimed is:

1. A mouse system comprising:
a plurality of temperature sensors;
a thermoelectric device;
a processor communicatively coupled to the temperature sensor and the thermoelectric device, the processor to:
receive a first input from a first temperature sensor of the plurality of temperature sensors wherein the first temperature sensor is positioned in proximity to a user's finger;
receive a second input from a second temperature sensor from the plurality of temperature sensors wherein the first temperature sensor is positioned in proximity to a user's palm;
activate the thermoelectric device based on the first input, the second input, and a machine learning model;
receive feedback from user responsive to the activation of the thermoelectric device; and
input feedback, first input, second input into the machine learning model.

2. The mouse system of claim 1, further comprising the processor to:
determine a temperature gradient between the first input and the second input;
input the temperature gradient, feedback, first input and second input into the machine learning model.

3. The mouse system of claim 1, further comprising:
an ambient temperature sensor of the plurality of temperature sensors; and
the processor further configured to:
receive an ambient temperature reading from the ambient temperature sensor;
input the ambient temperature, the feedback, first input and second input into the machine learning model.

4. The mouse system of claim 3, wherein the ambient temperature sensor comprises a connected smart thermostat.

5. The mouse system of claim 1 wherein the feedback comprises a temperature adjustment.

6. A method comprising:
receiving a first input from a first temperature sensor of a plurality of temperature sensors wherein the first temperature sensor is positioned in proximity to a users finger;
receiving a second input from a second temperature sensor from the plurality of temperature sensors wherein the first temperature sensor is positioned in proximity to a users palm;
receiving a third input from an ambient temperature sensor from the plurality of temperature sensors;
activating a thermoelectric device responsive to a machine learning model output wherein the first input, second input, and third input comprise a corresponding machine learning model input;
receiving feedback from user responsive to the activation of the thermoelectric device; and
inputting feedback, first input, second input, and third input into the machine learning model.

7. The method of claim 6, wherein the machine learning model output corresponds to a classification indicating a user comfort.

8. The method of claim 6, further comprising the processor to:
determine a temperature gradient between the first input and the second input;
input the temperature gradient, feedback, first input and second input into the machine learning model.

9. The method of claim 6, wherein the ambient temperature sensor comprises a connected smart thermostat.

10. The method of claim 6 wherein the feedback comprises a temperature adjustment of the thermoelectric device.

11. A computer readable medium comprising executable instructions that when executed cause a processor to:
receive a first input from a first temperature sensor of a plurality of temperature sensors wherein the first temperature sensor is positioned in proximity to a user's finger;
receive a second input from a second temperature sensor from the plurality of temperature sensors wherein the first temperature sensor is positioned in proximity to a user's palm;
determine a temperature gradient between the first input and the second input;
input the temperature gradient, first input and second input into the machine learning model;
activate a thermoelectric device based on the temperature gradient and a machine learning model;
receive feedback from user responsive to the activation of the thermoelectric device; and
input feedback and temperature gradient into the machine learning model.

12. The computer readable medium of claim 11, further comprising:
an ambient temperature sensor of the plurality of temperature sensors; and
executable instructions that when executed cause a processor to:
receive an ambient temperature reading from the ambient temperature sensor;
input the ambient temperature, the temperature gradient and feedback into the machine learning model.

13. The computer readable medium of claim 12, wherein the ambient temperature sensor comprises a connected smart thermostat.

14. The computer readable medium of claim 11 wherein the feedback comprises a temperature adjustment of the thermoelectric device.

15. The computer readable medium of claim 11, wherein the activation of the thermoelectric device further comprises:
input the temperature gradient into the machine learning model;
receive an output from the machine learning model, wherein the output corresponds to a classification;
determine whether the classification indicates a user discomfort; and
activate the thermoelectric device.
